(19) United States
(12) Patent Application Publication
(10) Pub. No.: US 2006/0122201 A1
(43) Pub. Date: Jun. 8, 2006

(54) Uracil-thioether

(76) Inventors: Niels Svenstrup, Wuppertal (DE); Alexander Kuhl, Hagen (DE); Dietmar Flubacher, Freiburg (DE); Michael Brands, Hamden, CT (US); Kerstin Ehlert, Velbert (DE); Christoph Ladel, Ivrea (IT); Michael Ottneder, Arlesheim (CH); Jorg Keldenich, Wuppertal (DE); Marcus Bauser, Wuppertal (DE)

Correspondence Address: Jeffrey M Greenman;Bayer Pharmaceuticals Corporation, 400 Morgan Lane, West Haven, CT, 06516, US

(21) Appl. No.: 10/506,006

(22) PCT Filed: Feb. 14, 2003
(86) PCT No.: PCT/EP03/01513

§ 371 Date: Dec. 23, 2005

(30) Foreign Application Priority Data
Feb. 27, 2002 (DE) 102 08 460.2

(51) Int. Cl. A61K 31/513; C07D 403/02; C07D 239/46

(52) U.S. Cl. 514/269; 544/309; 544/310

(57) ABSTRACT

The invention relates to uracil-thioethers, pharmaceutical compositions containing said compounds and to a method for the production thereof. The invention also relates to the use of said substances in the treatment of diseases in humans and animals.

The present invention relates to uracil thioethers, process for their preparation, pharmaceutical compositions including them, and the use thereof for the treatment of disorders in humans or animals.

Gram-positive eubacteria contain three different DNA polymerase exonucleases which are referred to as Pol 1, Pol 2 and Pol 3. Pol 3 is an enzyme which is necessary for the replicative synthesis of DNA.

The suitability of uracil derivatives for the treatment of bacterial infections has already been known for some time. Thus, WO 01/29010 describes 3-aminocarbonyl-substituted phenylaminouracils, WO 96/06614 describes 3-alkylidene-substituted uracils, WO 00/71523 describes 3-alkanoyloxyalkyluracils and WO 00/20556 describes uracils with zinc finger-active unit as antibacterial compounds. J Med. Chem., 1999, 42, 2035, Antimicro. Agents and Chemotherapy, 1999, 43, 1982 and Antimicro. Agents and Chemotherapy, 2000, 44, 2217 describe phenylaminouracils as antibacterial compounds.

Although further, structurally different agents with antibacterial activity are available on the market, it is normally possible for resistance to develop. Novel agents for better and effective therapy are therefore desirable.

One object of the present invention is therefore to provide novel compounds with identical or improved antibacterial effect for the treatment of antibacterial diseases in humans and animals.

It has surprisingly been found that derivatives of this class of compounds in which the uracil is substituted by a thioether have high antibacterial activity.

The present invention therefore relates to compounds of the general formula (I)

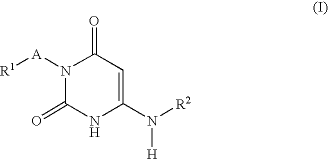

in which

R¹ is hydroxy, alkoxy, alkenyl, cycloalkyl, aryl, heterocyclyl or heteroaryl,
where R¹ equal to aryl may optionally be substituted by 1 to 3 substituents independently selected from the group of halogen, cyano, nitro, alkyl, alkoxy, alkanoyl, alkoxycarbonyl, amino, alkylamino, alkylsulfonyl, aminocarbonyl, alkylaminocarbonyl, aminosulfonyl and alkylaminosulfonyl,
and
where R¹ equal to heterocyclyl may optionally be substituted by 1 to 3 substituents independently selected from the group of oxo, alkyl, alkoxy, aryl, heteroaryl, alkanoyl and alkylsulfonyl,
in which aryl may optionally be substituted by 1 to 3 substituents independently selected from the group of halogen, nitro, alkyl and alkoxy,
and
where R¹ equal to heteroaryl may optionally be substituted by 1 to 3 substituents independently selected from the group of halogen, alkyl, alkoxy, alkylthio, cycloalkyl, aryl, oxo, alkanoyl, alkanoylamino, alkoxycarbonyl, amino, alkylamino, aminocarbonyl and alkylaminocarbonyl,

R² is a substituent of the following formula 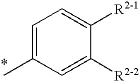
in which

R²⁻¹ and R²⁻² are selected independently of one another from the group of C₁-C₆-alkyl, C₂-C₆-alkenyl, C₂-C₆-alkynyl, C₃-C₆-cycloalkyl and halogen, or

R²⁻¹ and R²⁻² form together with the carbon atom to which they are bonded a C₃-C₆-cycloalkyl or heterocyclyl ring which may optionally be substituted by up to 3 halogen,

and

A is a C₃-C₆-alkanediyl chain in which one carbon atom is replaced by a sulfur atom, where at least 2 carbon atoms must be present between the sulfur atom in A and the nitrogen atom in the uracil ring, and where in the case where R¹ is equal to hydroxy or alkoxy at least 2 carbon atoms must be present between the sulfur atom in A and the oxygen atom in R¹,
and which is optionally substituted by up to 2 substituents selected from the group of hydroxy, alkoxy, oxo or amino.

The compounds of the invention may also be in the form of their salts, solvates or solvates of the salts.

The compounds of the invention may, depending on their structure, exist in stereoisomeric forms (enantiomers, diastereomers). The invention therefore relates to the enantiomers or diastereomers and respective mixtures thereof. The stereoisomerically pure constituents can be isolated in a known manner from such mixtures of enantiomers and/or diastereomers.

The invention also relates, depending on the structure of the compounds, to tautomers of the compounds.

Salts preferred for the purposes of the invention are physiologically acceptable salts of the compounds of the invention.

Physiologically acceptable salts of the compounds (I) include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, malic acid, citric acid, fumaric acid, maleic acid and benzoic acid.

Physiologically acceptable salts of the compounds (I) also include salts of conventional bases, such as by way of example and preferably alkali metal salts (e.g. sodium and potassium salts), alkaline earth metal salts (e.g. calcium and magnesium salts) and ammonium salts derived from ammonia or organic amines having 1 to 16 C atoms, such as by way of example and preferably ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, dihydroabietylamine, arginine, lysine, ethylenediamine and methylpiperidine.

Solvates refers for the purposes of the invention to those forms of the compounds which form a complex in the solid or liquid state through coordination with solvent molecules. Hydrates are a specific form of solvates in which the coordination takes place with water.

For the purposes of the present invention, the substituents have the following meaning unless specified otherwise:

Alkyl per se and “alk” and “alkyl” in alkoxy, alkylthio. alkylamino alkanoyl, alkanoylamino, alkylaminocarbonyl, alkylaminosulfonyl, alkoxycarbonyl, and alkylsulfonyl stand for a linear or branched alkyl radical having ordinarily from 1 to 6, preferably 1 to 4, particularly preferably 1 to 3, carbon atoms, by way of example and preferably methyl, ethyl, n-propyl, isopropyl, butyl, isobutyl, tert-butyl, n-pentyl and n-hexyl.

Cycloalkyl includes polycyclic saturated hydrocarbon radicals having up to 14 C atoms, namely monocyclic C₃-C₁₂-, preferably C₃-C₈-alkyl, such as, for example, cyclopropyl, cyclobutyl, cyclopentyl, cyclohexyl, cycloheptyl, cyclooctyl, cyclononyl, and polycyclic alkyl, i.e. preferably bicyclic and tricyclic, optionally spirocyclic C₇-C₁₄-alkyl, such as, for example, bicyclo[2.2.1]hept-1-yl, bicyclo[2.2.1]hept-2-yl, bicyclo[2.2.1]hept-7-yl, bicyclo[2.2.2]oct-2-yl, bicyclo[3.2.1]oct-2-yl, bicyclo[3.2.2]non-2-yl and adamantyl.

Alkenyl stands for a straight-chain or branched alkenyl radical having 2 to 6 carbon atoms. Preference is given to a straight-chain or branched alkenyl radical having 2 to 4, particularly preferably having 2 to 3 carbon atoms. Preferred examples which may be mentioned are: vinyl, allyl, n-prop-1-en-1-yl and n-but-2-en-1-yl.

Alkynyl stands for a straight-chain or branched alkenyl radical having 2 to 6 carbon atoms. Preference is given to a straight-chain or branched alkenyl radical having 2 to 4, particularly preferably having 2 to 3 carbon atoms. Preferred examples which may be mentioned are: n-prop-1-yn-1-yl and n-but-2-yn-1-yl.

Alkanediyl (alkylidene) stands for a carbon chain terminally substituted at both ends. Preference is given to saturated chains having 1 to 6 carbon atoms, in particular 2 to 4 carbon atoms.

Alkoxy stands by way of example and preferably for methoxy, ethoxy, n-propoxy, isopropoxy, tert-butoxy, n-pentoxy and n-hexoxy.

Alkylthio stands by way of example and preferably for methylthio, ethylthio, n-propylthio, isopropylthio, tert-butylthio, n-pentylthio and n-hexylthio.

Alkylamino stands for an alkylamino radical having one or two alkyl substituents (chosen independently of one another), by way of example and preferably methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

Alkanoyl stands by way of example and preferably for acetyl and propanoyl.

Alkanoylamino stands by way of example and preferably for acetylamino and ethylcarbonylamino.

Alkylaminocarbonyl stands for an alkylaminocarbonyl radical having one or two alkyl substituents (chosen independently of one another), by way of example and preferably methylaminocarbonyl, ethylaminocarbonyl, n-propylaminocarbonyl, isopropylaminocarbonyl, tert-butylaminocarbonyl, n-pentylaminocarbonyl, n-hexylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl, N-t-butyl-N-methylaminocarbonyl, N-ethyl-N-n-pentylaminocarbonyl and N-n-hexyl-N-methylaminocarbonyl.

Alkylaminosulfonyl stands for an alkylaminosulfonyl radical having one or two substituents (chosen independently of one another), by way of example and preferably methylaminosulfonyl, ethylaminosulfonyl, n-propylaminosulfonyl, isopropylaminosulfonyl, tert-butylaminosulfonyl, n-pentylaminosulfonyl, n-hexylaminosulfonyl, N,N-dimethylaminosulfonyl, N,N-diethylaminosulfonyl, N-ethyl-N-methylaminosulfonyl, N-methyl-N-n-propylaminosulfonyl, N-isopropyl-N-n-propylaminosulfonyl, N-t-butyl-N-methylaminosulfonyl, N-ethyl-N-n-pentylaminosulfonyl and N-n-hexyl-N-methylaminosulfonyl.

Alkoxycarbonyl stands by way of example and preferably for methoxycarbonyl, ethoxycarbonyl, n-propoxycarbonyl, isopropoxycarbonyl, tert-butoxycarbonyl, n-pentoxycarbonyl and n-hexoxycarbonyl.

Alkylsulfonyl stands by way of example and preferably for methylsulfonyl, ethylsulfonyl, n-propylsulfonyl, isopropylsulfonyl, tert-butylsulfonyl, n-pentylsulfonyl and n-hexylsulfonyl.

Aryl stands for a mono- to tricyclic aromatic, carbocyclic radical ordinarily having from 6 to 14 carbon atoms; by way of example and preferably phenyl, naphthyl and phenanthryl.

Heteroaryl stands for a 5- to 10-membered, in particular for a 5- to 6-membered, aromatic mono- or polycyclic heterocycle which is optionally bonded via a nitrogen atom and has up to 3 heteroatoms from the series S, O and/or N, for example for pyridyl, pyrimidyl, thienyl, furyl, pyrrolyl, thiazolyl, N-triazolyl, oxazolyl or imidazolyl. Preference is given to pyridyl, furyl, thiazolyl and N-triazolyl.

Heterocyclyl (heterocycle) stands for a mono- or polycyclic, heterocyclic radical which is optionally bonded via a nitrogen atom and has 3 to 11 ring atoms and up to 3, preferably 1, heteroatoms or hetero groups from the series N, O, S, SO, SO₂. In the case of polycyclic radicals, the rings may be fused (e.g. with a [0] bridge) or spiro-linked. 4- to 8-membered, in particular 5- and 6-membered, heterocyclyl is preferred. Mono- or bicyclic heterocyclyl is preferred. Monocyclic heterocyclyl is particularly preferred. N and O are preferred as heteroatoms. The heterocyclyl radicals may be saturated or partially unsaturated. The unsaturated representatives may comprise one or more double bonds in the ring or, in the case of polycyclic systems, be aromatic in one ring, such as, for example, benzoxazine. Saturated heterocyclyl radicals are preferred. The heterocyclyl radicals may be bonded via a carbon atom or a heteroatom. It may be formed from two substituent groups together with the nitrogen atom to which they are bonded. Particular preference is given to 5- to 7-membered, monocyclic saturated heterocyclyl radicals having up to two heteroatoms from the series O, N and S. Preferred examples which may be mentioned are: oxetanyl, pyrrolidinyl, pyrrolidinyl, pyrrolinyl, tetrahydrofuranyl, tetrahydrothienyl, pyranyl, piperidinyl, thiopyranyl, morpholinyl, perhydroazepinyl, thiomorpholinyl, piperazinyl, bicyclo[2.2.1]diazaheptyl.

Halogen stands for fluorine, chlorine, bromine and iodine.

A symbol * on a bond denotes the point of linkage in the molecule.

The radical definitions which are general or indicated in preferred ranges and are detailed above apply both to the final products of the formula (I) and correspondingly to starting materials and intermediates required for the preparation in each case.

The radical definitions indicated specifically in the particular combinations or preferred combinations of radicals are also replaced as desired by radical definitions of other combinations, irrespective of the combinations of radicals indicated in each case.

Preference is given for the purposes of the present invention to compounds of the general formula (I)

in which

R¹ is aryl, heterocyclyl or heteroaryl,
where R¹ equal to aryl may optionally be substituted by 1 to 2 substituents independently selected from the group of halogen, cyano, nitro, alkyl, alkoxy, alkanoyl and amino,
and
where R¹ equal to heterocyclyl may optionally be substituted by 1 to 3 substituents independently selected from the group of oxo, alkyl, alkoxy, aryl, heteroaryl, alkanoyl and alkylsulfonyl,
in which aryl may optionally be substituted by 1 to 2 substituents independently selected from the group of halogen, nitro, alkyl and alkoxy,
and
where R¹ equal to heteroaryl may optionally be substituted by 1 to 2 substituents independently selected from the group of halogen, alkyl, alkoxy, alkylthio, cycloalkyl, aryl, oxo, alkanoyl, alkanoylamino, alkoxycarbonyl, amino, alkylamino, aminocarbonyl and alkylaminocarbonyl,

R² is a substituent of the following formula 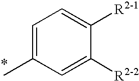
in which

R²⁻¹ and R²⁻² are independently of one another selected from the group of C₁-C₆-alkyl, C₂-C₆-alkenyl, C₂-C₆-alkynyl, C₃-C₆-cycloalkyl and halogen, or

R²⁻¹ and R²⁻² form together with the carbon atom to which they are bonded a C₃-C₆-cycloalkyl or heterocyclyl ring which may optionally be substituted by up to 3 halogen,

and

A is a C₃-C₆-alkanediyl chain in which one carbon atom is replaced by a sulfur atom, where at least 2 carbon atoms must be present between the sulfur atom in A and the nitrogen atom in the uracil ring, and where in the case where R¹ is equal to hydroxy or alkoxy at least 2 carbon atoms must be present between the sulfur atom in A and the oxygen atom in R¹,
and which is optionally substituted by up to 2 substituents selected from the group of hydroxy or oxo.

Preference is also given for the purposes of the present invention to compounds of the general formula (I) in which

R¹ is heteroaryl,
where the heteroaryl may optionally be substituted by 1 to 2 substituents independently selected from the group of halogen, alkyl, alkoxy, alkylthio, cycloalkyl, aryl, oxo, alkanoyl, alkanoylamino, alkoxycarbonyl, amino, alkylamino, aminocarbonyl and alkylaminocarbonyl,

and R² and A have the meaning indicated above.

Preference is also given for the purposes of the present invention to compounds of the general formula (I) in which

R¹ is thiadiazole or isoxazole,
where the thiadiazole or isoxazole may optionally be substituted by 1 to 2 substituents independently selected from the group of halogen, alkyl, alkoxy, alkylthio, cycloalkyl, aryl, oxo, alkanoyl, alkanoylamino, alkoxycarbonyl, amino, alkylamino, aminocarbonyl and alkylaminocarbonyl,

and R² and A have the meaning indicated above.

Preference is also given for the purposes of the present invention to compounds of the general formula (I) in which

R² is selected from the group consisting of:

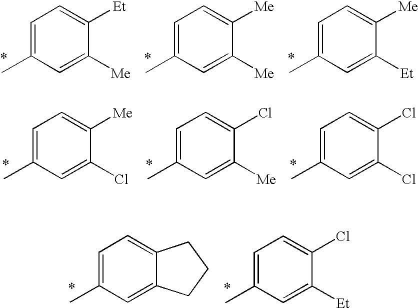

and R¹ and A have the meaning indicated above.

Preference is given for the purposes of the present invention among these in particular to compounds of the general formula (I) in which

R² is selected from the group consisting of:

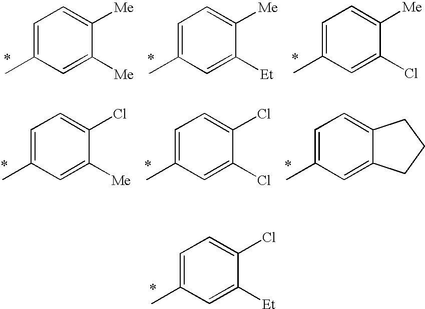

and R¹ and A have the meaning indicated above.

Preference is also given for the purposes of the present invention to compounds of the general formula (I) in which R² is a group

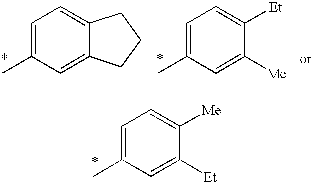

and R¹ and A have the meaning indicated above.

Preference is given for the purposes of the present invention among these in particular to compounds of the general formula (I) in which R² is a group

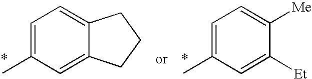

and R¹ and A have the meaning indicated above.

Preference is also given for the purposes of the present invention to compounds of the general formula (I) in which R¹-A is equal to

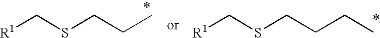

and R¹ and R² have the meaning indicated above.

Preference is also given for the purposes of the present invention to compounds of the general formula (I) in which R¹-A is equal to

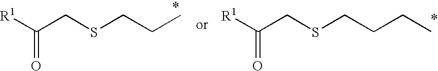

and R¹ and R² have the meaning indicated above.

The active ingredient may have systemic and/or local effects. For this purpose, it can be administered in a suitable manner such as, for example, by the oral, parenteral, pulmonary, nasal, sublingual, lingual, buccal, rectal, transdermal, conjunctival or otic route, or as implant.

The active ingredient can be administered in suitable administration forms for these administration routes.

Administration forms suitable for oral administration are known ones which deliver the active ingredient in a rapid and/or modified way, such as, for example, tablets (uncoated and coated tablets, e.g. tablets provided with coatings resistant to gastric juice or film-coated tablets), capsules, sugar-coated tablets, granules, pellets, powders, emulsions, suspensions and solutions.

Parenteral administration can take place with avoidance of an absorption step (intravenous, intraarterial, intracardiac, intraspinal or intralumbar) or with inclusion of an absorption (intramuscular, subcutaneous, intracutaneous, percutaneous or intraperitoneal). Administration forms suitable for parenteral administration include preparations for injection and infusion in the form of solutions, suspensions, emulsions, lyophilisates and sterile powders.

Examples suitable for the other administration routes are medicinal forms for inhalation (inter alia powder inhalers, nebulizers), nasal drops/solutions, sprays; tablets for lingual, sublingual or buccal administration, or capsules, suppositories, preparations for the eyes and ears, vaginal capsules, aqueous suspensions (lotions, shaking mixtures), lipophilic suspensions, ointments, creams, milk, pastes, dusting powders or implants.

The active ingredients can be converted in a manner known per se into the administration forms listed. This takes place with use of inert nontoxic, pharmaceutically suitable excipients. These include inter alia carriers (e.g. microcrystalline cellulose), solvents (e.g. liquid polyethylene glycols), emulsifiers (e.g. sodium dodecyl sulfate), dispersants (e.g. polyvinylpyrrolidone), synthetic and natural biopolymers (e.g. albumin), stabilizers (e.g. antioxidants such as ascorbic acid), colorants (e.g. inorganic pigments such as iron oxides) or masking tastes and/or odors.

It has generally proved advantageous for parental administration to administer amounts of about 0.001 to 10 mg/kg, preferably about 0.01 to 1 mg/kg, of body weight to achieve effective results. On oral administration the amount is about 0.01 to 500 mg/kg, preferably about 1 to 10 mg/kg, of body weight.

It may nevertheless be necessary where appropriate to deviate from the amounts mentioned, in particular as a function of the body weight, administration route, individual response to the active ingredient, type of preparation and time or interval over which administration takes place.

Particular preference is given to parenteral, especially intravenous, administration, e.g. as iv bolus injection (i.e. as single dose, e.g. by syringe), short infusion (i.e. infusion over a period of up to one hour) or long infusion (i.e. infusion over a period of more than one hour). The administered volume may in these cases be, depending on the specific conditions, between 0.5 to 30, in particular 1 to 20 ml on iv bolus injection, between 25 to 500, in particular 50 to 250 ml on short infusion and between 50 to 1000, in particular 100 to 500 ml on long infusion. It may for this purpose be advantageous for the active ingredient to be provided in solid form (e.g. as lyophilisate) to be dissolved in the dissolving medium only directly before administration.

(It is necessary in these cases for the administration forms to be sterile and pyrogen-free. They may be based on aqueous or mixtures of aqueous and organic solvents.

These include, for example, aqueous solutions, mixtures of aqueous and organic solvents (especially ethanol, polyethylene glycol (PEG) 300 or 400), aqueous solutions containing cyclodextrins or aqueous solutions containing emulsifiers (surface-active solubilizers, e.g. lecithin or Pluronic F 68, Solutol HS15, Cremophor). Aqueous solutions are preferred in this connection.

Formulations suitable for parenteral administration are those which are substantially isotonic and euhydric, e.g. those with a pH between 3 and 11, in particular 6 and 8, especially around 7.4.

Solutions for injection are packaged in suitable containers made of glass or plastic, e.g. in vials. These may have a volume of from 1 to 1000, in particular 5 to 50 ml. The solution can be removed directly therefrom and administered. In the case of a lyophilisate, it is dissolved in the vial by injecting a suitable solvent and is then removed.

Solutions for infusion are packaged in suitable containers made of glass or plastic, e.g. in bottles or collapsible plastic bags. These may have a volume of from 1 to 1000, in particular 50 to 500, ml.

The present invention further relates to a process for preparing the compounds of the general formula (I) characterized in that compounds of the general formula (II)

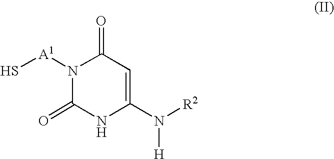

in which

R² has the meaning indicated above, and

A¹ is the part of A which is located between the sulfur atom and the uracil ring,

are reacted with compounds of the general formula (III)

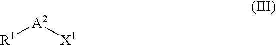

in which

R¹ has the meaning indicated above,

A² is the part of A which is located between the sulfur atom and the radical R¹,

and

X¹ is halogen, preferably chlorine, bromine or iodine.

The reaction takes place in inert solvents, where appropriate in the presence of a base, preferably in a temperature range from room temperature to 50° C. under atmospheric pressure.

Examples of inert solvents are halohydrocarbons such as methylene chloride, trichloromethane, tetrachloromethane, trichloroethane, tetrachloroethane, 1,2-dichloroethane or trichloroethylene, ethers such as diethyl ether, methyl tert-butyl ether, dioxane, tetrahydrofuran, glycol dimethyl ether or diethylene glycol dimethyl ether, or other solvents such as dimethylformamide, dimethylacetamide, 1,2-dimethoxyethane, dimethyl sulfoxide, acetonitrile or pyridine, with preference for tetrahydrofuran, dimethylformamide or methylene chloride.

Examples of bases are alkali metal carbonates such as, for example, sodium or potassium carbonate, or organic bases such as trialkylamines, e.g. triethylamine or diisopropylethylamine, or other bases such as, for example, N-methylmorpholine, N-methylpiperidine, 4-dimethylaminopyridine or 1,8-diazabicyclo(5.4.0)undec-7-ene, with preference for 1,8-diazabicyclo(5.4.0)undec-7-ene.

The compounds of the general formula (III) are known or can be synthesized by known processes from the appropriate precursors.

Compounds of the general formula (II) are prepared by reacting compounds of the general formula (IV)

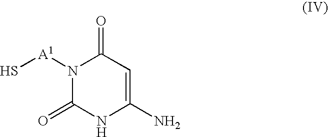

in which

A¹ has the meaning indicated above,

with compounds of the general formula (V)

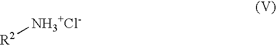

in which

R² has the meaning indicated above.

The reaction takes place where appropriate in inert solvents, in the presence of a base, preferably in a temperature range from 100° C. to 160° C. under atmospheric pressure.

Examples of inert solvents are ethers such as glycol dimethyl ether or diethylene glycol dimethyl ether, or other solvents such as dimethylformamide, or hydrocarbons such as benzene, ethylbenzene, xylene or toluene, with preference for reaction without solvent.

Examples of bases are organic bases such as trialkylamines, e.g. triethylamine or diisopropylethylamine, or other bases such as, for example, N-methylmorpholine, N-methylpiperidine, 4-dimethylaminopyridine or 1,8-diazabicyclo(5.4.0)undec-7-ene, with preference for diisopropylethylamine.

The compounds of the general formula (V) are known or can be synthesized by known processes from the appropriate precursors.

Compounds of the general formula (IV) are prepared by reacting compounds of the general formula (VI)

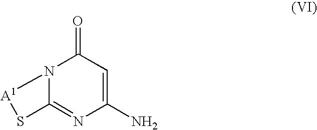

in which

A¹ has the meaning indicated above,

with aqueous alkali metal hydroxide solution.

The reaction preferably takes place in a temperature range of 100° C. under atmospheric pressure.

Examples of alkali metal hydroxides are sodium, potassium or lithium hydroxide, with preference for sodium hydroxide.

Compounds of the general formula (VI) are prepared by reacting compounds of the general formula (VII)

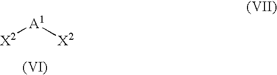

in which

A¹ has the meaning indicated above, and

X² is halogen, preferably bromine or iodine,

with

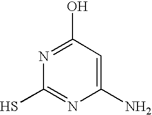

The reaction takes place in inert solvents, where appropriate in the presence of a base, preferably in a temperature range from room temperature to 60° C. under atmospheric pressure.

Examples of inert solvents are halohydrocarbons such as methylene chloride, trichloromethane, tetrachloromethane, trichloroethane, tetrachloroethane, 1,2-dichloroethane or trichloroethylene, ethers such as diethyl ether, methyl tert-butyl ether, dioxane, tetrahydrofuran, glycol dimethyl ether or diethylene glycol dimethyl ether, or other solvents such as dimethylformamide, dimethylacetamide, 1,2-dimethoxyethane, dimethyl sulfoxide, acetonitrile or pyridine, with preference for tetrahydrofuran, dimethylformamide or methylene chloride.

Examples of bases are alkali metal carbonates such as, for example, sodium, cesium or potassium carbonate, or organic bases such as trialkylamines, e.g. triethylamine or diisopropylethylamine, or other bases, such as, for example, N-methylmorpholine, N-methylpiperidine, 4-dimethylaminopyridine or 1,8-diazabicyclo(5.4.0)undec-7-ene, with preference for cesium carbonate.

The compounds of the general formula (VII) are known or can be synthesized by known processes from the appropriate precursors.

One preparation route is to be illustrated by way of example by scheme 1 below:

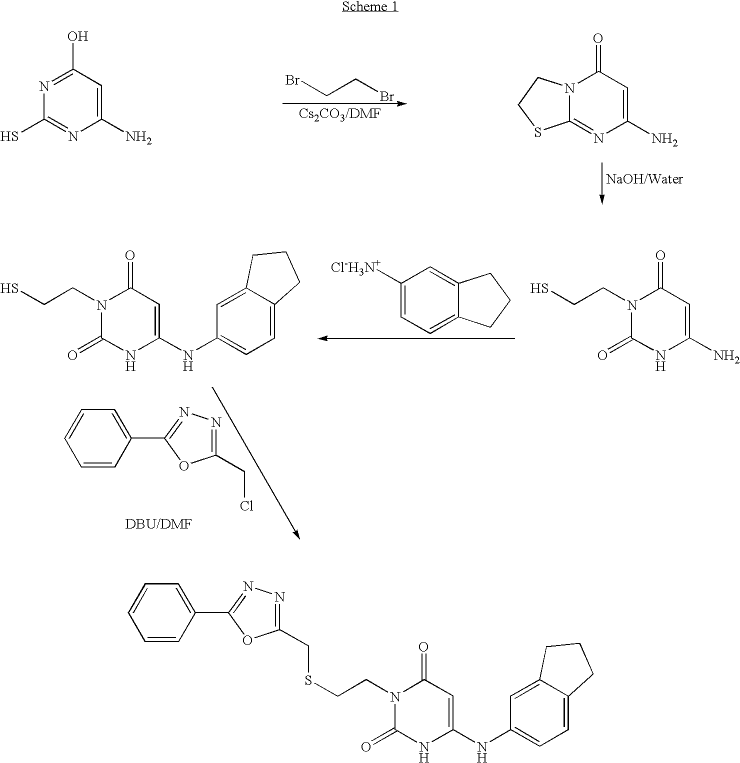

The compounds of the invention show a valuable range of pharmaceutical and pharmacokinetic effects which could not have been predicted. They are therefore suitable for use as medicaments for the treatment and/or prophylaxis of diseases in humans and animals.

The compounds of the invention are particularly effective against bacteria and bacteroid microorganisms, especially Gram-positive bacteria. They are therefore particularly suitable for the prophylaxis and chemotherapy of local and, where appropriate, systemic infections caused by these pathogens in human and veterinary medicine.

The compounds of the present invention are distinguished by a broad range of effects on Gram-positive bacteria, preferably including multiresistant microbes, especially staphylococci, pneumococci and enterococci, including vancomycin-resistant strains.

It is possible for example to treat and/or prevent local and/or systemic diseases caused by the following pathogens or by mixtures of the following pathogens:

Gram-positive cocci, e.g. staphylococci (Staph. aureus, Staph. epidermidis) and streptococci (Strept. agalactiae, Strept. faecalis, Strept. pneumoniae, Strept. pyogenes), and strictly anaerobic bacteria such as, for example, clostridium, also mycoplasmas (M. pneumoniae, M. hominis, M. urealyticum).

The above list of pathogens is merely by way of example and by no means to be regarded as restrictive. Examples of diseases which are caused by the mentioned pathogens or mixed infections, and can be prevented, improved or cured by the compounds of the invention, and which may be mentioned are:

infectious diseases in humans such as, for example, septic infections, bone and joint infections, skin infections, postoperative wound infections, abscesses, phlegmon, wound infections, infected bums, burn wounds, infections in the oral region, infections after dental operations, septic arthritis, mastitis, tonsillitis, genital infections and eye infections.

Apart from humans, it is also possible to treat bacterial infections in other species. Examples which may be mentioned are:

pig: sepsis, metritis-mastitis-agalactiae syndrome, mastitis;

ruminants (cattle, sheep, goats): sepsis, bronchopneumonia, mycoplasmosis, genital infections;

horse: bronchopneumonias, puerperal and postpuerperal infections;

dogs and cats: bronchopneumonia, dermatitis, otitis, urinary tract infections, prostatitis;

poultry (chickens, turkeys, quail, pigeons, ornamental birds and others): mycoplasmosis, chronic airway diseases, psittacosis.

It is likewise possible to treat bacterial diseases in the rearing and management of productive and ornamental fish, in which case the antibacterial spectrum is extended beyond the pathogens mentioned above to further pathogens such as, for example, brucella, campylobacter, listeria, erysipelothris, nocardia.

### A Assessment of the Pharmacological Activity

In Vitro Effect

The in vitro effect of the compounds of the invention can be shown in the following assays:

Cloning, Expression and Purification of Pol III from S. aureus

To clone polC with an N-terminal His tag, the structural gene polC is amplified from S. aureus genomic DNA with the aid of PCR. The primers SAPol 31 5′-GCGCCATATGGACAGAGCAACAAAAATTTAA-3′ and SAPolrev 5′-GCGCGGATCCTTACATATCAAATATCGAAA-3′ are used to introduce the NdeI and BamHI restriction cleavage sites respectively in front of and behind the amplified gene. After the PCR product which is 4300 bp in size has been digested with NdeI and BamHI, it is ligated into the vector pET15b (Novagen, USA), which has likewise been digested with NdeI and BamHI, and transformed into E. coli XL-1 Blue.

After transformation into E. coli BL21(DE3), the cells are cultivated for expression of PolC at 30° C. in LB medium with 100 μg/ml ampicillin until the OD₅₉₅ₙₘ is 0.5, cooled to 18° C. and, after addition of 1 mM IPTG, incubated for a further 20 hours. The cells are harvested by centrifugation, washed once in PBS with 1 mM PMSF and taken up in 50 mM NaH₂PO₄ pH 8.0, 10 mM imidazole, 2 mM β-mercaptoethanol, 1 mM PMSF, 20% glycerol. The cells are disrupted using a French press at 12,000 psi, the cell detritus is removed by centrifugation (27,000×g, 120 min, 4° C.) and the supernatant is stirred with an appropriate amount of Ni-NTA-agarose (from Quiagen, Germany) at 4° C. for 1 hour. After the gel matrix has been packed into a column it is washed with 50 mM NaH₂PO₄ pH 8.0, 2 mM β-mercaptoethanol, 20 mM imidazole, 10% glycerol, and the purified protein is then eluted with the same buffer containing 100 mM imidazole. The purified protein is mixed with 50% glycerol and stored at −20° C.

Determination of the IC₅₀ for Polymerase III

The activity of PolC is measured in an enzymatically coupled reaction, with the pyrophosphate formed during the polymerization being converted with the aid of ATP sulfurylase into ATP, which is detected using firefly luciferase. The reaction mixture contains, in a final volume of 50 μl, 50 mM Tris/Cl pH 7.5; 5 mM DTT, 10 mM MgCl₂, 30 mM NaCl, 0.1 mg/ml BSA, 10% glycerol, 20 μM each DATP, dTTP, dCTP, 2 U/ml activated calf thymus DNA (from Worthington, USA), 20 μM APS and 0.06 mM luciferin. The reaction is started by adding purified PolC in a final concentration of 2 nM and is incubated at 30° C. for 30 min. The amount of pyrophosphate formed is then converted into ATP by adding ATP sulfurylase (Sigma, USA) in a final concentration of 5 nM and incubating at 30° C. for 15 min. After addition of 0.2 nM firefly luciferase, the luminescence is measured in a luminometer for 60 s. The IC₅₀ is reported as the concentration of an inhibitor which leads to 50% inhibition of the enzymic activity of PolC.

TABLE A

| Example No. | IC₅₀ (μM) |
|---|---|
| 15 | 0.11 |
| 16 | 0.05 |
| 34 | 0.3 |
| 42 | 0.08 |
| 47 | 0.4 |

Determination of the Minimum Inhibitory Concentrations (MIC) for a Number of Microbes

The MIC values for various bacterial strains (S. aureus, S. pneumoniae, E. faecalis) are carried out using the microdilution method in BHI broth. The bacterial strains are cultured overnight in BHI broth (staphylococci) or BHI broth+10% bovine serum (streptococci, enterococci). The test substances are tested in a concentration range from 0.5 to 256 μg/ml. After serial dilution of the test substances, the microtiter plates are inoculated with the test microbes. The microbe concentration is about 1×10⁶ microbes/ml of suspension. The plates are incubated at 37° C. under 8% CO₂ (for streptococci, enterococci) for 20 h. The MIC is recorded as the lowest concentration at which visible growth of the bacteria is completely inhibited.

In Vivo Effect

The suitability of the compounds of the invention for treating bacterial infections can be shown in the following animal models:

Systemic Infection with S. aureus 133

S. aureus 133 cells are cultured overnight in BH broth. The overnight culture is diluted 1:100 in fresh BH broth and amplified for 3 hours. The bacteria, which are in the logarithmic phase of growth, are spun down and washed 2× with buffered physiological saline (303). Then a cell suspension is adjusted in a photometer (model LP 2W from Dr. Lange, Germany) to an extinction of 50 units in 303. After a dilution step (1:15), the suspension is mixed 1:1 with a 10% strength mucinis suspension. 0.25 ml of this infection solution is administered ip per 20 g mouse. This corresponds to a cell count of approximately 1×10E⁶ microbes/mouse. The ip therapy takes place 30 minutes after the infection. Female CFW1 mice are used for the infection experiment. The survival of the animals is recorded for 6 days.

### B. EXAMPLES

Abbreviations

aq. aqueous

DBU 1,8-diazabicyclo(5.4.0)undec-7-ene

DMSO dimethyl sulfoxide

DMF dimethylformamide

eq. equivalent

ESI electrospray ionization (in MS)

h hour

HPLC high pressure, high performance liquid chromatography

LC-MS coupled liquid chromatography-mass spectroscopy

MeCN acetonitrile

MS mass spectroscopy

NMR nuclear magnetic resonance spectroscopy

RP-HPLC reverse phase HPLC

RT room temperature

Rₜ retention time (in HPLC)

LC-MS Methods

Method 1

Instrument: Micromass Quattro LCZ, HP1100; column: symmetry C18, 50 mm×2.1 mm, 3.5 μm; eluent B: water +0.1% formic acid, eluent A: acetonitrile +0.1% formic acid; gradient: 0.0 min 10% A→4.0 min 90% A→6.0 min 90% A; oven: 40° C., flow rate: 0.5 ml/min, UV detection: 208-400 nm

Method 2

Instrument: Micromass Platform LCZ, HP1100; column: symmetry C18, 50 mm×2.1 mm, 3.5 μm; eluent B: water +0.1% formic acid, eluent A: acetonitrile +0.1% formic acid; gradient: 0.0 min 10% A→4.0 min 90% A→6.0 min 90% A; oven: 40° C., flow rate: 0.5 mvmin, UV detection: 208-400 nm

Method 3

Instrument: Finnigan MAT 900S, TSP: P4000,AS3000,UV30000HR; column: symmetry C18, 150 mm×2.1 mm, 5.0 μm; eluent C: water, eluent B: water +0.6 g of 35% HCI, eluent A: acetonitrile; gradient: 0.0 min 2% A, 49% B, 49% C→2.5 min 95% A, 2.5% B, 2.5% C→5.5 min 2% A, 49% B, 49% C; oven: 70° C., flow rate: 1.2 ml/min, UV detection: 210 nm

Starting Compounds

### Example I

### 7-Amino-2,3-dihydro-5H-[1,3]thiazolo[3,2-a]pyrimidin-5-one

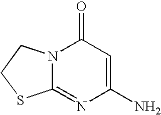

27.22 g (144.9 mmol) of 1,2-dibromoethane and 85.84 g (263.46 mmol) of cesium carbonate are suspended in 125 ml of dimethylformamide under argon. Then, over the course of 30 minutes, 18.86 g (131.73 mmol) of 6-amino-4-hydroxy-2-mercaptopyrimidine are added. The mixture is stirred at room temperature for 4 hours and then heated at 60° C. overnight. The reaction solution is cooled and concentrated in vacuo. The residue is mixed with 100 ml of water. The mixture is stirred for 10 minutes and then left to stand for 10 minutes. The precipitate is filtered off with suction, washed twice with 25 ml of water and dried under high vacuum. 15.8 g (71% of theory) of product are obtained.

LC-MS (method 2): Rₜ=0.53 min

MS (ESIpos): m/z=170 (M+H)⁺

### Example II

### 8-Amino-3,4-dihydro-2H,6H-pyrimido[2,1-b][1,3]thiazin-6-one

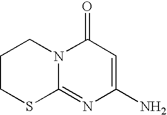

Preparation takes place as for Example I from 11.26 g (69.85 mmol) of 6-amino-4-hydroxy-2-mercaptopyrimidine, 16.22 g (80.32 mmol) of 1,3-dibromopropane and 45.52 g (136.69 mmol) of cesium carbonate. 12 g (93% of theory) of product are obtained.

LC-MS (method 1): Rₜ=0.64 min

MS (ESIpos): m/z=184 (M+H)⁺

### Example III

### 6-Amino-3-(2-sulfanylethyl)-2,4(1H,3H)pyrimidinedione

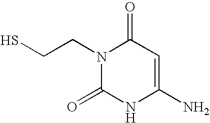

15.5 g (9.16 mmol) of the compound from Example I and 14.66 g (366.42 mmol) of sodium hydroxide are suspended together in 100 ml of water and heated to reflux for 2 hours. A clear solution results. The reaction solution is cooled to 0° C. and neutralized with concentrated hydrochloric acid. The mixture is stirred at 0° C. for one hour. The precipitate is then filtered off, washed twice with 20 ml of water and diethyl ether each time and dried in a vacuum oven at 60° C. overnight. 13.93 g (81% of theory) of the product are obtained.

¹H-NMR (200 MHz, DMSO-d₆) δ=3.79 (t, 2H), 4.54 (d, 1H), 6.23 (s, 2H), 10.42 (s, 1H)

LC-MS (method 2): Rₜ=0.66 min

MS (ESIpos): m/z=188 (M+H)⁺

### Example IV

### 6-Amino-3-(3-sulfanylpropyl)-2,4(1H,3H)pyrimidinedione

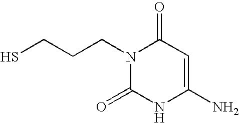

Preparation takes place as for Example III from 4.3 g (23.47 mmol) of the compound from Example II and 3.75 g (98.87 mmol) of sodium hydroxide. 1.52 g (32% of theory) of product are obtained.

LC-MS (method 1): Rₜ=0.94 min

MS (ESIpos): m/z=218 (M+H)⁺

### Example V

### 6-(2,3-Dihydro-1H-inden-5-ylamino)-3-(2-sulfanylethyl)-2,4(1H,3H)pyrimidinedione

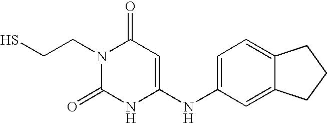

3 g (16.02 mmol) of the compound from Example III, 3.2 g (24.04 mmol) of 5-aminoindane and 4.08 g (24.04 mmol) of 5-aminoindane hydrochloride are heated together at 160° C. for 6 hours. After the reaction solution has cooled to room temperature, it is stirred up with 50% ethanol and the precipitate is filtered off with suction. The latter is then washed once again with 20 ml of 50% ethanol and twice with 20 ml of diethyl ether each time and dried under high vacuum. 4.71 g (97% of theory) of product are obtained.

¹H-NMR (300 MHz, DMSO-d₆) δ=2.02 (quintet, 2H), 2.59 (q, 2H), 2.84 (q, 4H), 3.83 (t, 2H), 4.71 (s, 1H), 6.95 (d, 1H), 7.06 (s, 1H), 7.22 (d, 1H), 8.17 (s, 1H), 10.43 (s, 1H)

LC-MS (method 2): Rₜ=3.53 min

MS (ESIpos): m/z=304 (M+H)⁺

### Example VI

### 6-[(3-Ethyl-4-methylphenyl)amino]-3-(2-sulfanylethyl)-2,4(1H,3H)pyrimidinedione

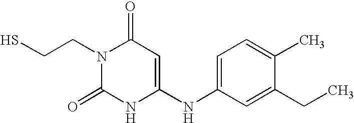

Preparation takes place as for Example V from 3 g (16.02 mmol) of the compound from Example III, 5.78 g (33.65 mmol) of 3-ethyl-4-methylaniline hydrochloride and 2.93 ml (16.82 mmol) of N,N-diisopropylethylamine. 4.83 g (98% of theory) of product are obtained.

¹H-NMR (300 MHz, DMSO-d₆) δ=1.14 (t, 3H), 2.24 (s, 3H), 2.53-2.63 (m, 4H), 3.83 (t, 2H), 4.71 (s, 1H), 6.91-6.98 (m, 2H), 7.14 (d, 1H), 8.1 (s, 1H), 10.41 (s, 1H),

LC-MS (method 3): Rₜ=2.39 min

MS (ESIpos): m/z=306 (M+H)⁺

### Example VII

### 6-[(3-Ethyl-4-methylphenyl)amino]-3-(3-sulfanylpropyl)-2,4(1H,3H)-pyrimidinedione

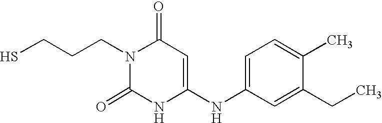

Preparation takes place as for Example V from 2.98 g (14.81 mmol) of the compound from Example IV, 7.63 g (44.42 mmol) of 3-ethyl-4-methylaniline hydrochloride and 3.87 ml (22.21 mmol) of N,N-diisopropylethylamine. 4.25 g (90% of theory) of product are obtained.

¹H-NMR (200 MHz, DMSO-d₆) δ=1.13 (t, 3H), 1.7-1.83 (m, 2H), 2.23 (s, 3H), 3.77 (t, 2H), 4.72 (s, 1H), 6.97 (s, 1H), 7.05 (dd, 2H), 8.15 (s, 1H), 10.48 (s, 1H), 4H also under the DMSO peak

LC-MS (method 1): Rₜ=3.7 min

MS (ESIpos): m/z=32−(M+H)⁺

### PREPARATION EXAMPLES

### Example 1

### 6-(2,3-Dihydro-1H-inden-5-ylamino)-3-(2-{[(5-phenyl-1,3,4-oxadiazol-2-yl)methyl]sulfanyl}ethyl)-2,4(1H,3H)pyrimidinedione

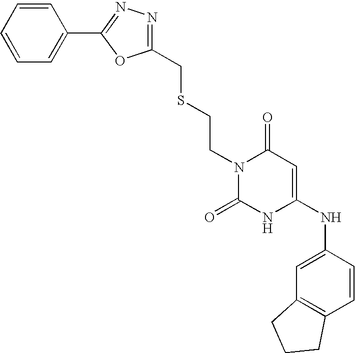

40 mg (0.13 mmol) of the compound from Example V and 28.23 mg (0.15 mmol) of 2-(chloromethyl)-5-phenyl-1,3,4-oxadiazole are introduced into 3 ml of dimethylformamide. Then 40.14 g (0.26 mmol) of 1,8-diazabicyclo(5.4.0)undec-7-ene are added, and the mixture is stirred at room temperature for 3 hours. The reaction solution is concentrated in vacuo. The residue is taken up in dichloromethane and washed once with 1N hydrochloric acid and once with saturated sodium chloride solution. The organic phase is dried over sodium sulfate and concentrated in vacuo. Drying under high vacuum results in 56.8 mg (84% of theory) of product.

LC-MS (method 2): Rₜ=3.97 min

MS (ESIpos): m/z=462 (M+H)⁺

The examples listed in the following table can be prepared in analogy to the methods described above from the appropriate starting compounds.

| Exp. | | |
|---|---|---|
| No. | Structure | Analytical data |
| 2 | 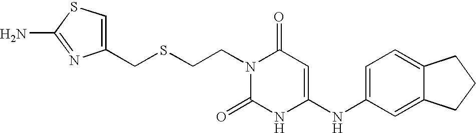 | LC-MS (method 1): Rt = 2.66 min MS (ESIpos): m/z =416 (M + H) |
| 3 | 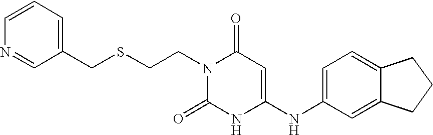 | LC-MS (method 2): Rt = 2.81 min MS (ESIpos): m/z =395 (M + H) |
| 4 | 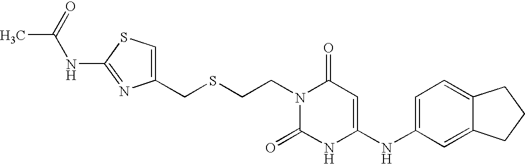 | LC-MS (method 1): Rt = 3.47 min MS (ESIpos): m/z =458 (M + H) |
| 5 | 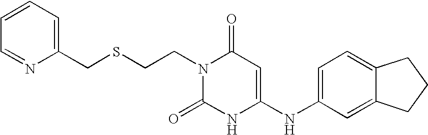 | LC-MS (method 2): Rt = 2.89 min MS (ESIpos): m/z =395 (M + H) |
| 6 | 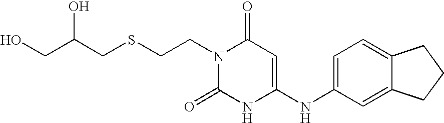 | LC-MS (method 1): Rt = 3.04 min MS (ESIpos): m/z =378 (M + H) |
| 7 | 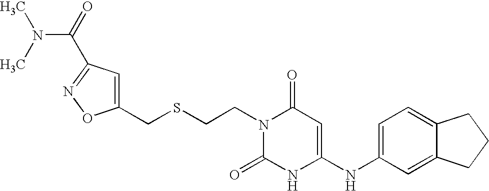 | LC-MS (method 3): Rt = 2.35 min MS (ESIpos): m/z =456 (M + H) |
| 8 | 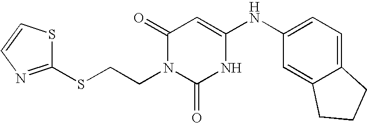 | LC-MS (method 2): Rt = 3.69 min MS (ESIpos): m/z =387 (M + H) |
| 9 | 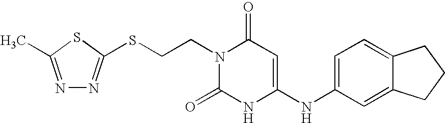 | LC-MS (method 1): Rt = 3.41 min MS (ESIpos): m/z =402 (M + H) |
| 10 | 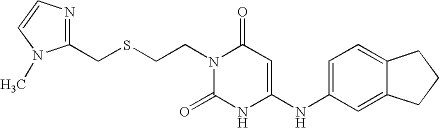 | LC-MS (method 1): Rt = 2.52 min MS (ESIpos): m/z =398 (M + H) |
| 11 | 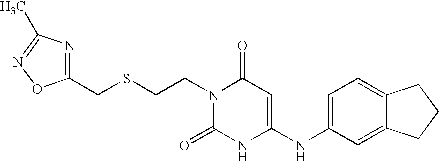 | LC-MS (method 1): Rt = 3.56 min MS (ESIpos): m/z =400 (M + H) |
| 12 | 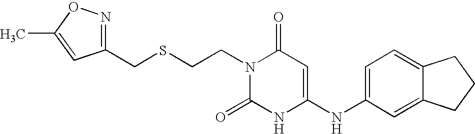 | LC-MS (method 1): Rt = 3.73 min MS (ESIpos): m/z =399 (M + H) |
| 13 | 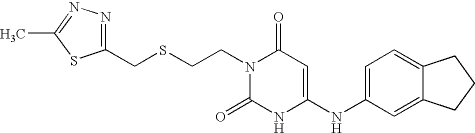 | LC-MS (method 1): Rt = 3.42 min MS (ESIpos): m/z =416 (M + H) |
| 14 | 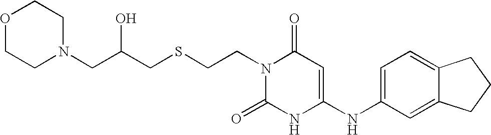 | LC-MS (method 2): Rt = 2.57 min MS (ESIpos): m/z =447 (M + H) |
| 15 | 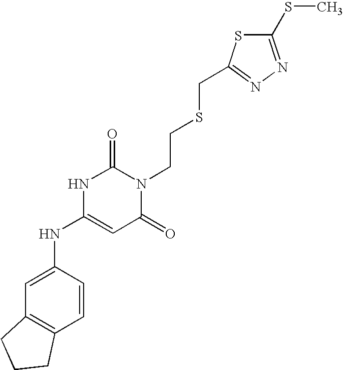 | LC-MS (method 2): Rt = 3.77 min MS (ESIpos): m/z =448 (M + H) |
| 16 | 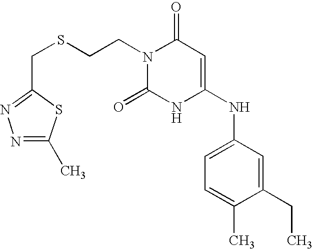 | LC-MS (method 2): Rt = 3.58 min MS (ESIpos): m/z =418 (M + H) |
| 17 | 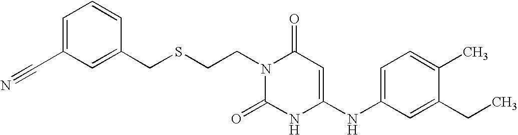 | LC-MS (method 2): Rt = 3.87 min MS (ESIpos): m/z =450 (M + H) |
| 18 | 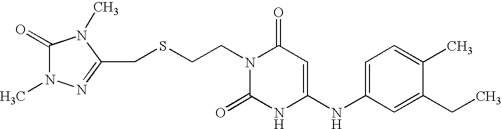 | LC-MS (method 1): Rt = 4.12 min MS (ESIpos): m/z =421 (M + H) |
| 19 | 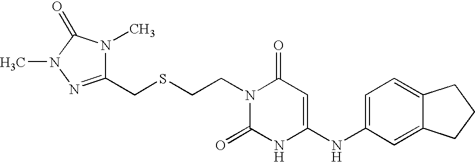 | 1H-NMR(200 MHz, DMSO-d6) delta =1.12(t, 3H), 2.21(s, 3H), 2.67(t, 2H), 3.15(s, 3H), 3.27(s, 3H), 3.75(s, 2H), 3.91(t, 2H), 4.74(s, 1H), 6.98-7.12(m, 3H), 8.76(s, 1H), 2H also underneath the DMSO |
| 20 | 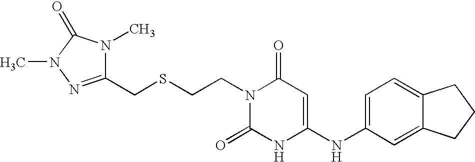 | 1H-NMR(200 MHz, CDCl3) delta = 2.05(quintet, 2H), 2.75-2.9(m, 6H), 3.23(s, 3H), 3.38(s, 3H), 3.57(s, 2H), 3.95(t, 2H), 5.05(s, 1H), 6.98(s, 1H), 6.99(dd, 2H) |
| 21 | 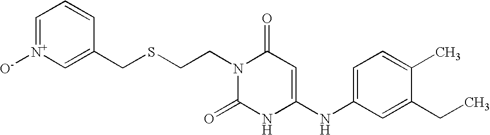 | LC-MS (method 3): Rt = 2.49 min MS (ESIpos): m/z =413(M + H) |
| 22 | 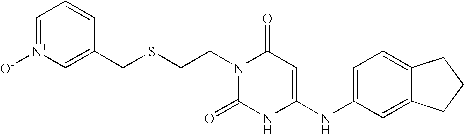 | LC-MS (method 3): Rt = 2.51 min MS (ESIpos): m/z =411 (M + H) |
| 23 | 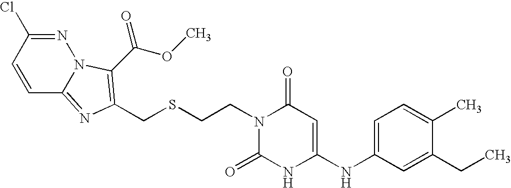 | LC-MS (method 3): Rt = 2.44 min MS (ESIpos): m/z =529 (M + H) |
| 24 | 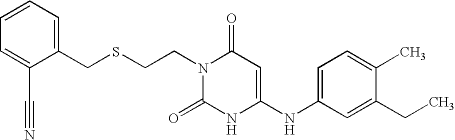 | LC-MS (method 3): Rt = 2.80 min MS (ESIpos): m/z =421 (M + H) |
| 25 | 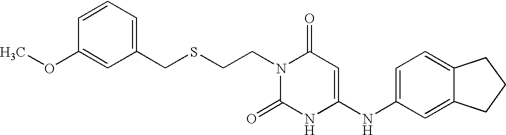 | LC-MS (method 3): Rt = 2.82 min MS (ESIpos): m/z =424 (M + H) |
| 26 | 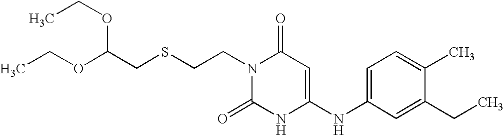 | LC-MS (method 3): Rt = 2.64 min MS (ESIpos): m/z =422 (M + H) |
| 27 | 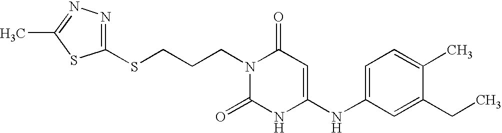 | LC-MS (method 3): Rt = 2.44 min MS (ESIpos): m/z =418 (M + H) |
| 28 | 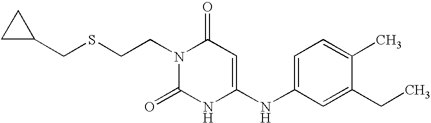 | LC-MS (method 1): Rt = 4.12 min MS (ESIpos): m/z =360 (M + H) |
| 29 | 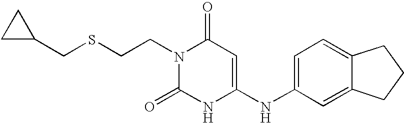 | |
| 30 | 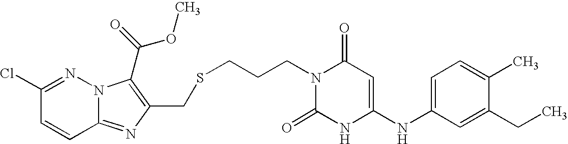 | LC-MS (method 1): Rt = 3.88 min MS (ESIpos): m/z =543 (M + H) |
| 31 | 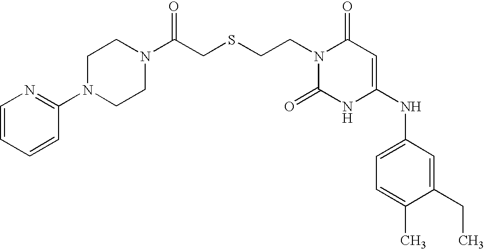 | LC-MS (method 1): Rt = 2.85 min MS (ESIpos): m/z =509 (M + H) |
| 32 | 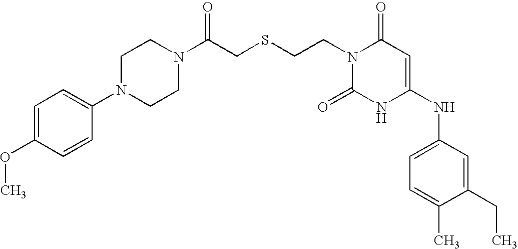 | LC-MS (method 1): Rt = 3.79 min MS (ESIpos): m/z =538 (M + H) |
| 33 | 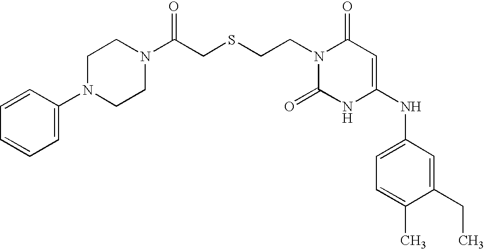 | LC-MS (method 1): Rt = 4.01 min MS (ESIpos): m/z =508 (M + H) |
| 34 | 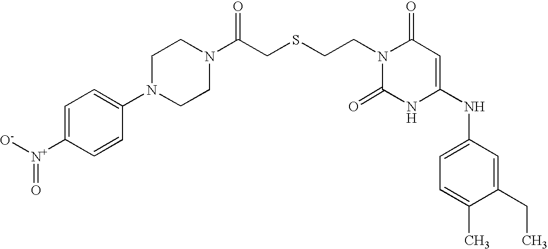 | LC-MS (method 1): Rt = 3.97 min MS (ESIpos): m/z =553 (M + H) |
| 35 | 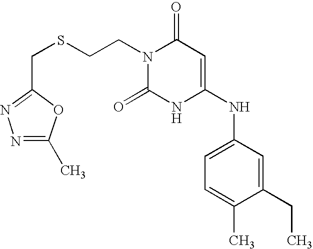 | LC-MS (method 1): Rt = 3.47 min MS (ESIpos): m/z =402 (M + H) |
| 36 | 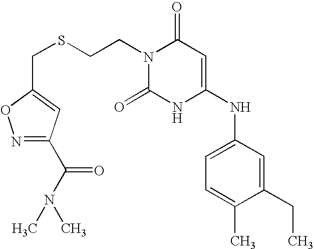 | LC-MS (method 1): Rt = 3.69 min MS (ESIpos): m/z =458 (M + H) |
| 37 | 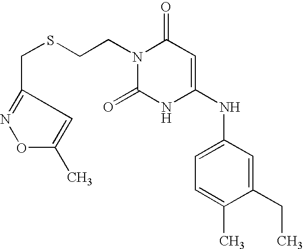 | LC-MS (method 1): Rt = 3.86 min MS (ESIpos): m/z =401 (M + H) |
| 38 | 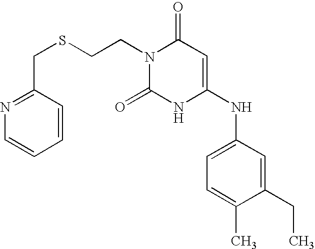 | LC-MS (method 1): Rt = 3.02 min MS (ESIpos): m/z =397 (M + H) |
| 39 | 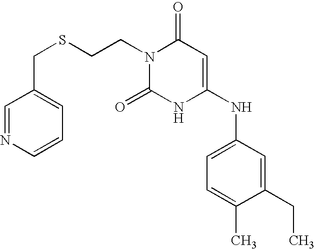 | LC-MS (method 1): Rt = 2.92 min MS (ESIpos): m/z =397 (M + H) |
| 40 | 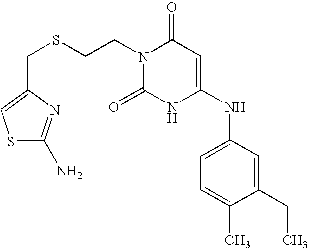 | LC-MS (method 1): Rt = 2.81 min MS (ESIpos): m/z =418 (M + H) |
| 41 | 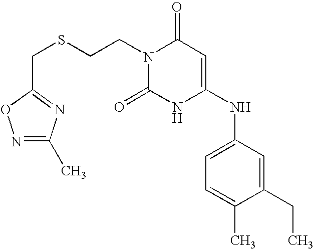 | LC-MS (method 1): Rt = 3.7 min MS (ESIpos): m/z =402 (M + H) |
| 42 | 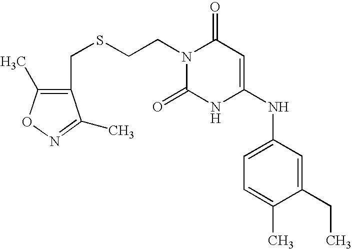 | LC-MS (method 1): Rt = 3.89 min MS (ESIpos): m/z =415 (M + H) |
| 43 | 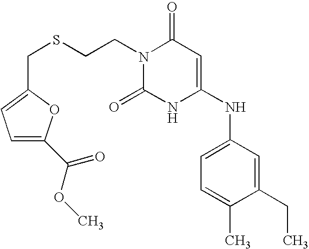 | LC-MS (method 1): Rt = 4.01 min MS (ESIpos): m/z =444 (M + H) |
| 44 | 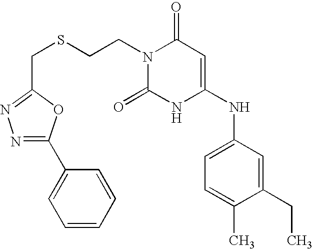 | LC-MS (method 1): Rt = 4.01 min MS (ESIpos): m/z =464 (M + H) |
| 45 | 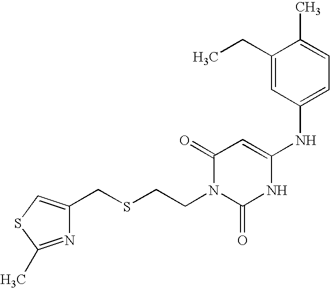 | LC-MS (method 1): Rt = 3.74 min MS (ESIpos): m/z =417 (M + H) |
| 46 | 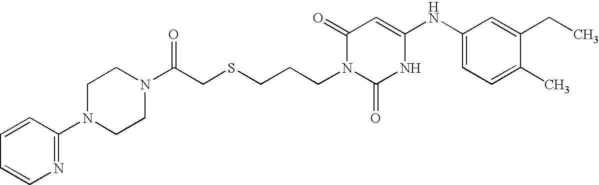 | LC-MS (method 1): Rt = 2.89 min MS (ESIpos): m/z =523 (M + H) |
| 47 | 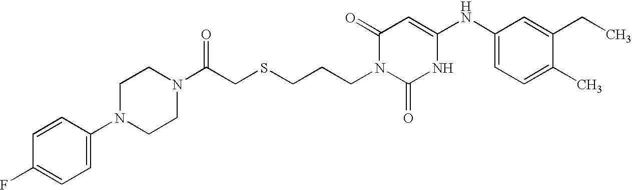 | LC-MS (method 1): Rt = 4.10 min MS (ESIpos): m/z =540 (M + H) |
| 48 | 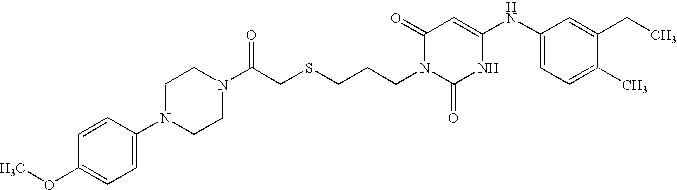 | LC-MS (method 1): Rt = 3.85 min MS (ESIpos): m/z =552 (M + H) |
| 49 | 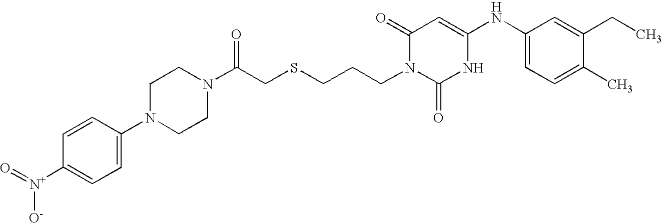 | LC-MS (method 1): Rt = 4.03 min MS (ESIpos): m/z =567 (M + H) |
| 50 | 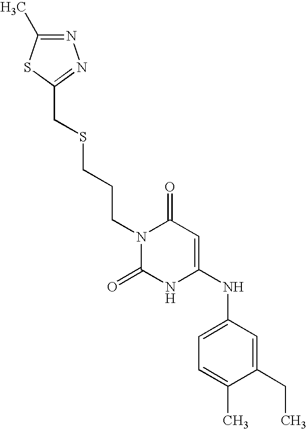 | LC-MS (method 1): Rt = 3.60 min MS (ESIpos): m/z =432 (M + H) |
| 51 | 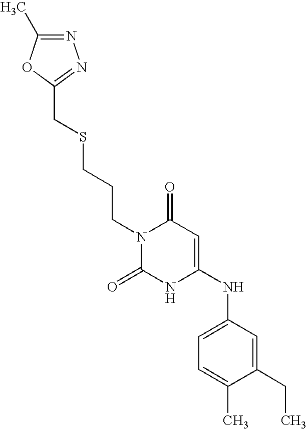 | LC-MS (method 1): Rt = 3.51 min MS (ESIpos): m/z =416 (M + H) |
| 52 | 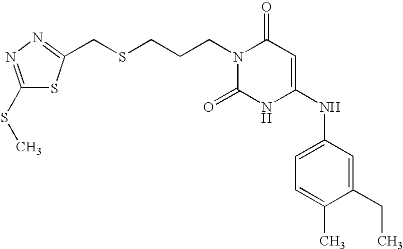 | LC-MS (method 1): Rt = 3.88 min MS (ESIpos): m/z =464 (M + H) |
| 53 | 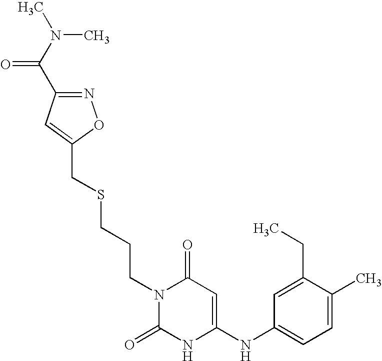 | LC-MS (method 1): Rt = 3.71 min MS (ESIpos): m/z =472 (M + H) |
| 54 | 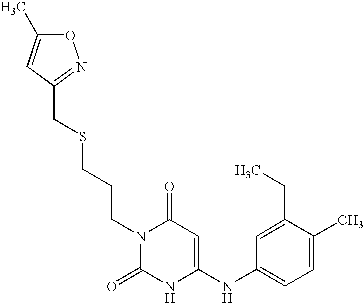 | LC-MS (method 1): Rt = 3.9 min MS (ESIpos): m/z =415 (M + H) |
| 55 | 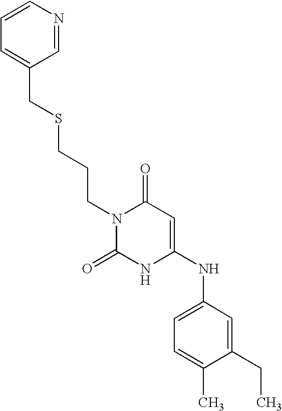 | LC-MS (method 1): Rt = 2.97 min MS (ESIpos): m/z =411 (M + H) |
| 56 | 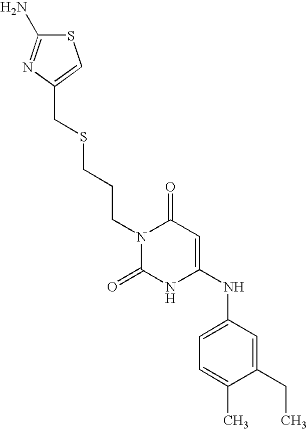 | LC-MS (method 1): Rt = 2.85 min MS (ESIpos): m/z =432 (M + H) |
| 57 | 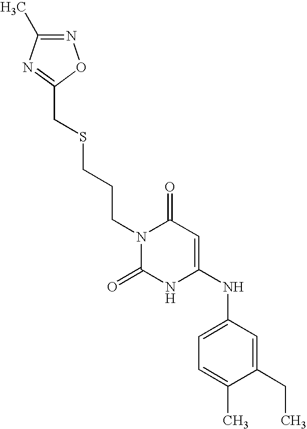 | LC-MS (Method 1): Rt = 3.75 min MS (ESIpos): m/z =416 (M + H) |
| 58 | 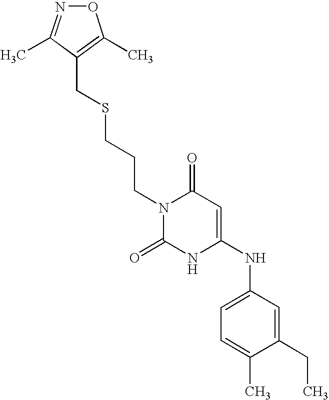 | LC-MS (Method 1): Rt = 3.94 min MS (ESIpos): m/z =429 (M + H) |
| 59 | 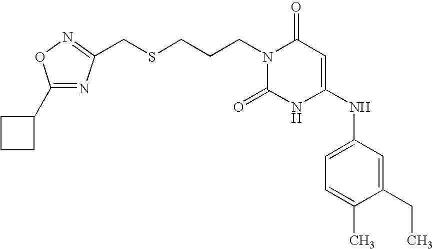 | LC-MS (Method 1): Rt = 4.14 min MS (ESIpos): m/z =456 (M + H) |
| 60 | 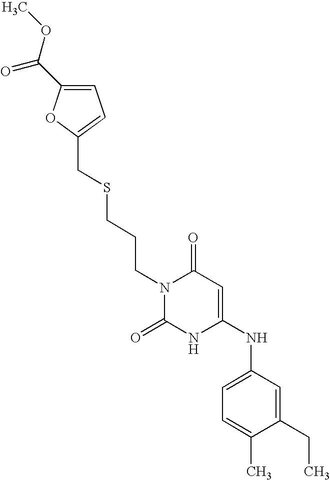 | LC-MS (Method 1): Rt = 4.04 min MS (ESIpos): m/z =458 (M + H) |
| 61 | 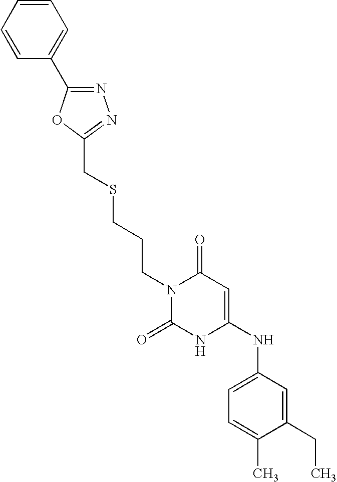 | LC-MS (Method 1): Rt = 4.05 min MS (ESIpos): m/z =478 (M + H) |
| 62 | 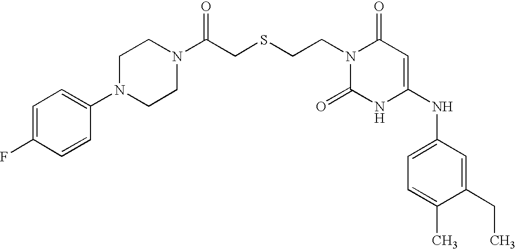 | LC-MS (Method 1): Rt = 4.05 min MS (ESIpos): m/z =526 (M + H) |
| 63 | 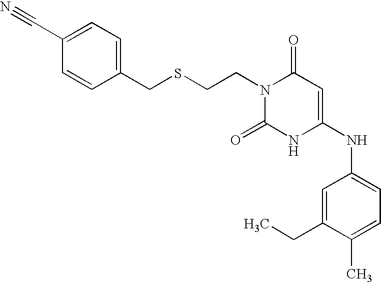 | LC-MS (Method 1): Rt = 4.13 min MS (ESIpos): m/z =421 (M + H) |
| 64 | 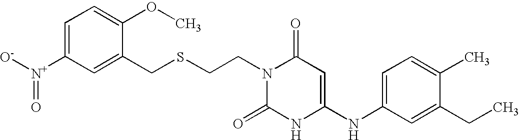 | LC-MS (Method 1): Rt = 4.25 min MS (ESIpos): m/z =471 (M + H) |
| 65 | 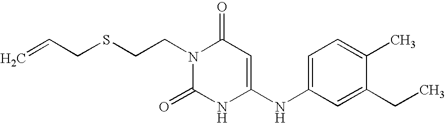 | LC-MS (Method 1): Rt = 3.97 min MS (ESIpos): m/z =346 (M + H) |
| 66 | 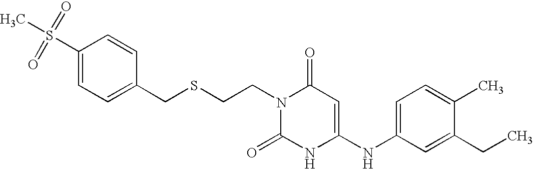 | LC-MS (Method 1): Rt = 3.82 min MS (ESIpos): m/z =474 (M + H) |
| 67 | 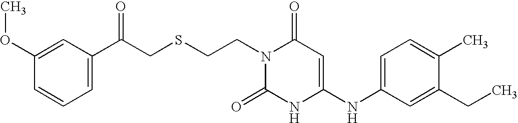 | LC-MS (Method 1): Rt = 4.12 min MS (ESIpos): m/z =454 (M + H) |
| 68 | 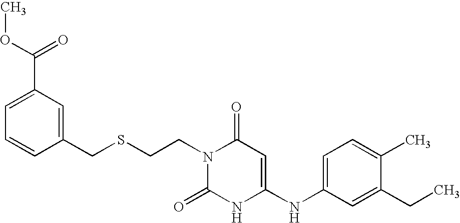 | LC-MS (Method 1): Rt = 4.23 min MS (ESIpos): m/z =454 (M + H) |
| 69 | 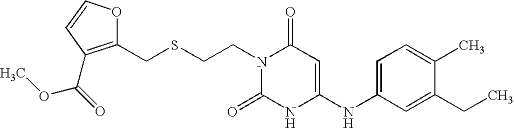 | LC-MS (Method 1): Rt = 4.04 min MS (ESIpos): m/z =444 (M + H) |
| 70 | 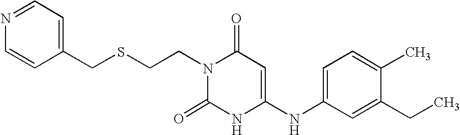 | LC-MS (Method 1): Rt = 2.74 min MS (ESIpos): m/z =397 (M + H) |

### C. Exemplary Embodiments of Pharmaceutical Compositions

The substances of the invention can be converted into pharmaceutical preparations in the following ways:

Tablet:

Composition:

100 mg of the compound of Example 1, 50 mg of lactose (monohydrate), 50 mg of corn starch (native), 10 mg of polyvinylpyrolidone (PVP 25) (from BASF, Ludwigshafen, Germany) and 2 mg of magnesium stearate.

Tablet weight 212 mg, diameter 8 mm, radius of curvature 12 mm.

Production:

The mixture of active ingredient, lactose and starch is granulated with a 5% strength solution (m/m) of the PVP in water. The granules are dried and then mixed with the magnesium stearate for 5 min. This mixture is compressed in a conventional tablet press (see above for format of tablet). A compressive force of 15 kN is used as guideline value for the compression.

Suspension Which Can Be Administered Orally:

Composition:

1000 mg of the compound of Example 1, 1000 mg of ethanol (96%), 400 mg of Rhodigel (xanthan gum from FMC, Pennsylvania, USA) and 99 g of water.

10 ml of oral suspension correspond to a single dose of 100 mg of the compound of the invention.

Production:

The Rhodigel is suspended in ethanol, and the active ingredient is added to the suspension. The water is added while stirring. Stirring is continued for about 6 h until the swelling of the Rhodigel is complete.

Solution Which Can Be Administered Intravenously:

Composition:

1 mg of the compound of Example 1, 15 g of polyethylene glycol 400 and 250 g of water for injection.

Production:

The compound of Example 1 is dissolved with polyethylene glycol 400 in the water with stirring. The solution is sterilized by filtration (pore diameter 0.22 μm) and dispensed under aseptic conditions into heat-sterilized infusion bottles. These are closed with infusion stoppers and crimped caps.

1. A compound of the formula 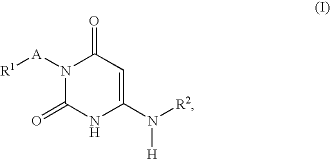
in which
R¹ is hydroxy, alkoxy, alkenyl, cycloalkyl, aryl, heterocyclyl or heteroaryl,
where R¹ equal to aryl may optionally be substituted by 1 to 3 substituents independently selected from the group of halogen, cyano, nitro, alkyl, alkoxy, alkanoyl, alkoxycarbonyl, amino, alkylamino, alkylsulfonyl, aminocarbonyl, alkylaminocarbonyl, aminosulfonyl and alkylaminosulfonyl,
and
where R¹ equal to heterocyclyl may optionally be substituted by 1 to 3 substituents independently selected from the group of oxo, alkyl, alkoxy, aryl, heteroaryl, alkanoyl and alkylsulfonyl,
in which aryl may optionally be substituted by 1 to 3 substituents independently selected from the group of halogen, nitro, alkyl and alkoxy,
and
where R¹ equal to heteroaryl may optionally be substituted by 1 to 3 substituents independently selected from the group of halogen, alkyl, alkoxy, alkylthio, cycloalkyl, aryl, oxo, alkanoyl, alkanoylamino, alkoxycarbonyl, amino, alkylamino, aminocarbonyl and alkylaminocarbonyl,
R² is a substituent of the following formula 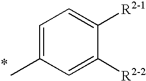
in which
R²⁻¹ and R²⁻² are selected independently of one another from the group of C₁-C₆-alkyl, C₂-C₆-alkenyl, C₂-C₆-alkynyl, C₃-C₆-cycloalkyl and halogen, or
R²⁻¹ and R²⁻² form together with the carbon atom to which they are bonded a C₃-C₆-cycloalkyl or heterocyclyl ring which may optionally be substituted by up to 3 halogen,
and
A is a C₃-C₆-alkanediyl chain in which one carbon atom is replaced by a sulfur atom, where at least 2 carbon atoms must be present between the sulfur atom in A and the nitrogen atom in the uracil ring, and where in the case where R¹ is equal to hydroxy or alkoxy at least 2 carbon atoms must be present between the sulfur atom in A and the oxygen atom in R¹, and which is optionally substituted by up to 2 substituents selected from the group of hydroxy, alkoxy, oxo or amino.

2. The compound of the formula (I) as claimed in claim 1, in which
R¹ is aryl, heterocyclyl or heteroaryl,
where R¹ equal to aryl may optionally be substituted by 1 to 2 substituents independently selected from the group of halogen, cyano, nitro, alkyl, alkoxy, alkanoyl and amino,
and
where R¹ equal to heterocyclyl may optionally be substituted by 1 to 3 substituents independently selected from the group of oxo, alkyl, alkoxy, aryl, heteroaryl, alkanoyl and alkylsulfonyl,
in which aryl may optionally be substituted by 1 to 2 substituents independently selected from the group of halogen, nitro, alkyl and alkoxy,
and
where R¹ equal to heteroaryl may optionally be substituted by 1 to 2 substituents independently selected from the group of halogen, alkyl, alkoxy, alkylthio, cycloalkyl, aryl, oxo, alkanoyl, alkanoylamino, alkoxycarbonyl, amino, alkylamino, aminocarbonyl and alkylaminocarbonyl,
R² is a substituent of the following formula 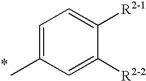
in which
R²⁻¹ and R²⁻² are independently of one another selected from the group of C₁-C₆-alkyl, C₂-C₆-alkenyl, C₂-C₆-alkynyl, C₃-C₆-cycloalkyl and halogen, or
R²⁻¹ and R²⁻² form together with the carbon atom to which they are bonded a C₃-C₆-cycloalkyl or heterocyclyl ring which may optionally be substituted by up to 3 halogen,
and
A is a C₃-C₆-alkanediyl chain in which one carbon atom is replaced by a sulfur atom, where at least 2 carbon atoms must be present between the sulfur atom in A and the nitrogen atom in the uracil ring, and where in the case where R¹ is equal to hydroxy or alkoxy at least 2 carbon atoms must be present between the sulfur atom in A and the oxygen atom in R¹, and which is optionally substituted by up to 2 substituents selected from the group of hydroxy or oxo.

3. The compound of the formula (I) as claimed in claim 1, in which
R¹ is heteroaryl,
where R¹ equal to heteroaryl may optionally be substituted by 1 to 2 substituents independently selected from the group of halogen, alkyl, alkoxy, alkylthio, cycloalkyl, aryl, oxo, alkanoyl, alkanoylamino, alkoxycarbonyl, amino, alkylamino, aminocarbonyl and alkylaminocarbonyl.

4. The compound of the formula (I) as claimed in claim 1, in which
R² is selected from the group

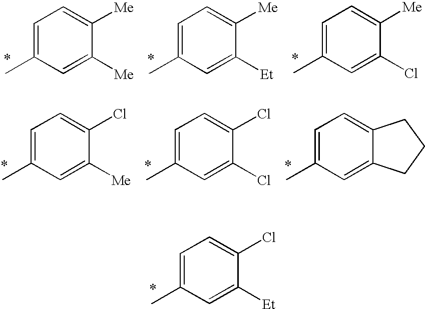

5. The compound of the formula (I) as claimed in claim 1, in which
R¹-A is equal to

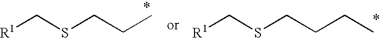

6. The compound of the formula (I) as claimed in claim 1, in which
R¹-A is equal to

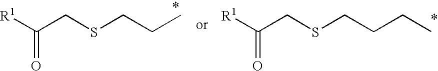

7. A process for preparing the compounds of the formula (I) by reacting compounds of the formula 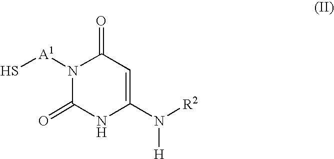
in which
R² has the meaning indicated in claim 1, and
A¹ is the part of A which is located between the sulfur atom and the uracil ring,
with compounds of the general formula (III) 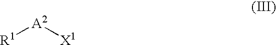
in which
R¹ has the meaning indicated in claim 1,
A² is the part of A which is located between the sulfur atom and the radical R¹,
and
X¹ is halogen.

8. (canceled)

9. A pharmaceutical composition comprising at least one compound as claimed in claim 1 in combination with at least one pharmaceutically acceptable, pharmaceutically suitable carrier or excipient.

10. (canceled)

11. (canceled)

12. A method for controlling bacterial infections in humans and animals, comprising administering to a patient in need thereof an antibacterially effective amount of at least one compound as claimed in claim 1.
